SEQUENCE LISTING (13 sequences)

SEQ ID NO: 1 - protein, 133 aa, Homo sapiens [sequence omitted]

SEQ ID NO: 2 - protein, 129 aa, Homo sapiens [sequence omitted]

SEQ ID NO: 3 - protein, 158 aa, Homo sapiens
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHHHHHHH

SEQ ID NO: 4 - protein, 126 aa, Homo sapiens
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 5 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 503 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 7 - protein, 121 aa, Homo sapiens
MHHHHHHNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 133 aa, Homo sapiens [sequence omitted]

SEQ ID NO: 9 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 5 aa, Homo sapiens
GGGGS

SEQ ID NO: 11 - protein, 370 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 358 aa, Homo sapiens
MHHHHHHNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 370 aa, Homo sapiens
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0009656 A1
(43) Pub. Date: Jan. 9, 2025

(54) ULTRA PH-SENSITIVE MICELLES ENCAPSULATING CYTOKINES FOR TREATMENT OF CANCER

(71) Applicant: The Board of Regents of The University of Texas System, Austin, TX (US)

(72) Inventors: Jinming GAO, Dallas, TX (US); Gang HUANG, Dallas, TX (US); Zhichen SUN, Dallas, TX (US); Qiang FENG, Dallas, TX (US); Wei LI, Dallas, TX (US)

(73) Assignee: The Board of Regents of The University of Texas System, Austin, TX (US)

(21) Appl. No.: 18/710,555

(22) PCT Filed: Nov. 15, 2022
(86) PCT No.: PCT/US2022/079899

§ 371 Date: May 15, 2024

Related U.S. Application Data
(60) Provisional application No. 63/279,594, filed on Nov. 15, 2021.

(51) Int. Cl. A61K 9/107; A61K 38/20; A61K 47/34; A61K 47/64; A61K 47/68; C08F 293/00

(52) U.S. Cl. CPC A61K 9/107; A61K 38/20; A61K 47/34; A61K 47/642; A61K 47/68; C08F 293/005

(57) ABSTRACT

Ultra pH sensitive (UPS) micelles encapsulating cytokines are provided that provide lower toxicity and higher efficacy compositions and methods for treating cancer with those compositions.

This application claims the benefit of priority to U.S. Provisional Application No. 63/279,594, filed on Nov. 15, 2021, the entire contents of which are hereby incorporated by reference.

This invention was made with government support under Grant No. 1U54CA244719 awarded by the National Institute of Health. The government has certain rights in the invention.

Pursuant to 37 C.F.R. 1.821 (c), a sequence listing is submitted herewith as an ST.26 xml file named “UTFD.P3990WO Sequence Listing.xml”, created on Nov. 15, 2022 and having a size of 15 kilobytes. The content of the aforementioned file is hereby incorporated by reference in its entirety.

### BACKGROUND

Interleukin 2 (IL-2) is a critical component in T cell homeostasis and immune regulation. IL-2 has shown good tumor rejection efficacy in combination with other immunotherapies such as adoptive cell transfer, checkpoint inhibition, etc. However, the pleiotropic function of IL-2 in stimulating regulatory T cells, as well as its short serum half-life (3.7 min+/−0.8 min) and severe toxicity limit the application of IL-2 in cancer therapy.

Treatment of cancer using interleukin 2 (IL-2) has been investigated since the early 1980s starting with Cetus Corporation's recombinant version of IL-2, first branded as Adesleukin and later Proleukin. In 1992, the FDA approved Proleukin for treatment of metastatic renal carcinoma. A modified, recombinant version of IL-2 called Teceleukin, with a methionine added at its N-terminal has been developed by Roche. A version of IL-2 called Bioleukin, with a methionine added at its N-terminal and residue 125 replaced with alanine, has been developed by GSK. Dozens of clinical trials have been conducted using recombinant or purified IL-2, alone or in combination with other drugs, or using cell therapies in which cells are taken from patients, activated with IL-2, and then reinfused.

Proleukin has shown modest efficacy, including 6-7% complete remission and 8-10% partial response. The contrary functions of IL-2 require balancing tumor-killing (requiring high doses) over toxicity and immunosuppression (requiring low doses) and managing dose limiting toxicities. Frequent and severe broad spectrum serious adverse events (SAEs) limit patient eligibility. The short half-life of IL-2 necessitates frequent dosing, such as 20 infusions over 3 weeks. Although many have tried to identify a novel/modified IL-2 with an improved therapeutic window, those efforts have not resulted in a clinically validated candidate to date.

Past attempts to improve IL-2 efficacy have included fusing a moiety to the IL-2, such as polyethylene glycol (PEG), cytokine, or receptor subunit moieties, as well as fusing proteins such as antibodies to direct selective binding to a cell. Other approaches have involved administration in combination with a cofactor that downregulates toxic pathways, e.g., Treg depletion. Approaches involving PEGylation may include selective binding and improved half-life, but do not provide tumor specificity or reduction of exposure to normal tissues.

There remains a need to deliver IL-2 safely and effectively to tumors with an improved therapeutic profile.

### SUMMARY OF THE INVENTION

The present invention relates to a micelle composition encapsulating a cytokine active agent comprising:
(i) a block copolymer of Formula (I), or a pharmaceutically acceptable salt, solvate, or hydrate thereof:

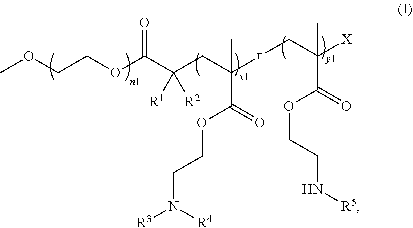

wherein:
   n₁ is an integer from 40-500;
   x₁ is an integer from 4-150;
   y₁ is an integer from 0-10;
   X is a halogen, —OH, or —C(O)OH;
   R¹ and R² are each independently hydrogen or optionally substituted C₁-C₆ alkyl;
   R³ and R⁴ are each independently an optionally substituted C₁-C₆ alkyl, C₃-C₁₀ cycloalkyl or aryl;
   or R³ and R⁴ are taken together with the corresponding nitrogen to which they are attached form an optionally substituted 5 to 7-membered ring;
   R⁵ is hydrogen or —C(O)CH₃; and
   (ii) the cytokine active agent, wherein the cytokine active agent is an interleukin or interleukin-Fc construct.

The block copolymer may include a hydrophobic polymer segment selected from:

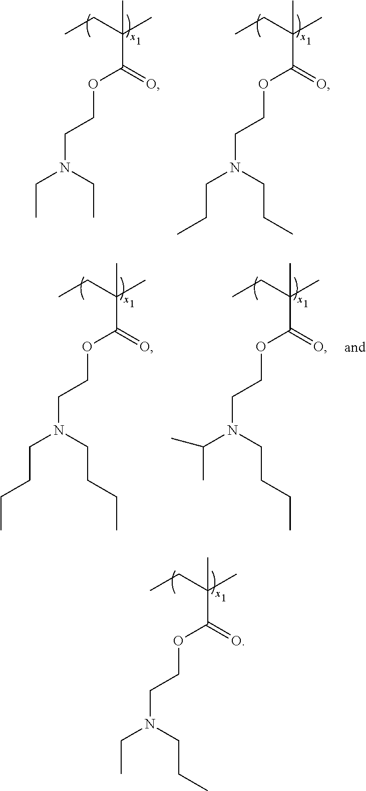

In one aspect, the hydrophobic segment is selected from:

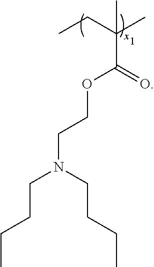

The composition above may further be a composition where n₁ is an integer from 115-340, x₁ is an integer from 30-90, and/or y₁ is 0. In this case, the molecular weight of n1 ranges from approximately 5 k Daltons to 15 k Daltons.

In one aspect the composition includes a cytokine that is an interleukin. The interleukin may be selected from IL-2, IL-4, IL-7, IL-9, IL-10, IL-12, IL-15, or IL-21. The cytokine is preferably IL-2, IL-15, or IL-21. In one preferred aspect, the cytokine is IL-21. The cytokine may also be a fusion protein. In one aspect, the cytokine is an interleukin fused to an antibody fragment through a linker moiety. In one aspect, the antibody fragment is an IgG Fc antibody fragment. The cytokine may include an amino acid comprising an amino acid sequence of SEQ ID NOs: 1, 2, 3, 4, 5, 6, 7, 8, or 9 or a homolog that is at least 95% identical to the amino acid sequence of SEQ ID NOs: 1, 2, 3, 4, 5, 6, 7, 8, or 9. In one aspect, the Fc region comprises an amino acid sequence of SEQ ID NO: 11. In another aspect, the cytokine active agent comprises a linker of SEQ ID: 10. In another aspect, the cytokine active agent comprises an amino acid of SEQ ID NOs: 11, 12, or 13 or a homolog that is at least 95% identical to the amino acid sequence of SEQ ID NOs: 11, 12, or 13. In one preferred aspect, the cytokine active agent comprises an amino acid of SEQ ID NOs: 11.

In another aspect, the invention involves a method of treatment of a solid tumor comprising administering a micelle composition encapsulating a cytokine active agent described above to a patient in need thereof. The method may comprise administering a composition that the comprises a pharmaceutically acceptable excipient. The method preferably comprises administering the composition through intravenous (IV) route of administration. However, the composition may also be administered intratumorally, by for example intratumoral injection.

### BRIEF DESCRIPTION OF THE DRAWINGS

FIG. 1 shows a depiction of IL-2 receptor biology indicating the requirement of high local concentration of IL-2.

FIG. 2 shows polymeric micelles of ultra pH sensitive (UPS) polymers working as an ON/OFF switch for Fc-fused IL-2 cytokines.

FIG. 3A shows predicted PDBA binding site at the IL-2Ra binding domain.

FIG. 3B shows predicted PDBA binding at other IL-2 sites.

FIG. 4A shows the IL-2 loading within PDBA polymer-based UPS micelles having different hydrophilic segment molecular weight for the PDBA.

FIG. 4B shows the IL-2 loading within PDBA polymer-based UPS micelles having different hydrophobic segment molecular weight for the PDBA.

FIG. 4C shows the IL-2 loading within PDBA polymer-based UPS micelles having different hydrophilic and hydrophobic segment molecular weight for pH ranging between 4.7-7.4 for the PDBA.

FIG. 5 shows pH titration of PDBA based UPS polymers having different hydrophilic and hydrophobic segment molecular weight.

FIG. 6 shows the tumor volume as a measurement of antitumor efficacy and relative body weight as a measure of toxicity of IL-2Fc encapsulated micelles compared to free IL-2Fc and IgG.

FIG. 7A shows toxicity biomarkers for various IL-2Fc encapsulated micelles based on low dose treatment of an MC38 hot tumor.

FIG. 7B shows toxicity of various IL-2Fc encapsulated micelles based on low dose treatment of an MC38 hot tumor.

FIG. 8A shows tumor inhibition for hot tumor, low dose treatment window.

FIG. 8B shows CR/MCP-1@24 hr for hot tumor, low dose treatment window.

FIG. 9A shows the treatment schedule and IL-2 ELISA 4-6 h, 24 h for B16F10 cold tumor, high dose treatment.

FIG. 9B shows the tumor volume as a measure of antitumor efficacy for IL-2Fc encapsulated micelles compared to free IL-2Fc and IgG.

FIG. 9C shows the relative body weight as a measure of toxicity for IL-2Fc encapsulated micelles compared to free IL-2Fc and IgG.

FIG. 10A shows toxicity of various IL-2Fc encapsulated micelles based on high dose treatment of a B16F10 cold tumor.

FIG. 10B shows toxicity of various IL-2Fc encapsulated micelles based on high dose treatment of a B16F10 cold tumor.

FIG. 11 shows tumor inhibition for B16F10 k, cold tumor, for various polymer IL-2Fc compositions.

FIG. 12 shows the percentage loading of IL-2, IL-2Fc, IL-4, IL-7, IL-9, IL-10, IL-12, IL-15 and IL-21 in a UPS polymer in accordance with the invention.

FIG. 13 shows the effect of hydrophobic group side chain on IL-2 loading.

FIG. 14A shows the instability of IL-2-IR800 encapsulation in a DSPE-PEG5 k copolymer formulation in water and FBS solutions.

FIG. 14B shows the stability of IL-2 encapsulation in a PDBA 10 k60 copolymer formulation according to an aspect of the invention in both water and FBS solutions.

### DETAILED DESCRIPTION

Provided herein are pharmaceutical compositions that include ultra pH sensitive (UPS) polymers that form micelles which have been found to non-covalently encapsulate certain cytokines. The micelles of the present invention, including for example polyethylene glycol-poly 2-(dibutylamino)ethyl methacrylate (PEG-PDBA), have been shown to have particular affinity for encapsulating interleukins, and in particular IL-2 and IL-2 fused to the Fc region of the IgG antibody. A particular affinity for encapsulation was shown for IL-2, IL-15 and IL-21, relative to other interleukins, such as IL-4, IL-7, IL 9, IL-10, and IL-12.

The pharmaceutical compositions described herein typically include a block copolymer having a hydrophilic and hydrophobic segment that allow formation of micelles.

### 1. Compositions

In certain embodiments, provided herein is a pharmaceutical composition comprising:
(i) a block copolymer of Formula (I), or a pharmaceutically acceptable salt, solvate, or hydrate thereof:

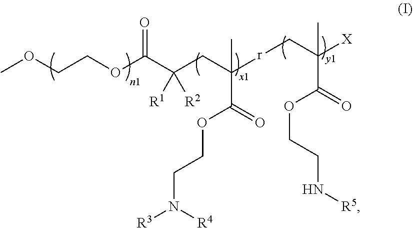

wherein:
   n₁ is an integer from 40-500;
   x₁ is an integer from 4-150;
   y₁ is an integer from 0-10;
   X is a halogen, —OH, or —C(O)OH;
   R¹ and R² are each independently hydrogen or optionally substituted C₁-C₆ alkyl;
   R³ and R⁴ are each independently an optionally substituted C₁-C₆ alkyl, C₃-C₁₀ cycloalkyl or aryl;
   or R³ and R⁴ are taken together with the corresponding nitrogen to which they are attached form an optionally substituted 5 to 7-membered ring;
   R⁵ is hydrogen or —C(O)CH₃; and
   (ii) the cytokine active agent, wherein the cytokine active agent is an interleukin or interleukin-Fc construct.

### (i) Block Copolymers

In some embodiments, the pharmaceutical composition comprises a block copolymer of Formula (I), or a pharmaceutically acceptable, salt, solvate, or hydrate thereof.

In some embodiments of Formula (I), R¹ and R² are each independently an optionally substituted C₁-C₆ alkyl. In some embodiments, R¹ and R² are each independently —CH₃, —CH₂CH₃, —CH₂CH₂CH₃, or —CH₂CH₂CH₂CH₃. In some embodiments, R¹ and R² are each independently —CH₃. In some embodiments, R¹ and R² are each independently hydrogen.

In some embodiments of Formula (I), the R³ and R⁴ are each independently an optionally substituted C₁-C₆ alkyl. In some embodiments, the alkyl is a straight chain or a branch alkyl. In some embodiments, the alkyl is a straight chain alkyl. In some embodiments, R³ and R⁴ are each independently —CH₂CH₃, —CH₂CH₂CH₃, or —CH₂CH₂CH₂CH₃. In some embodiments, R³ and R⁴ are each independently —CH₂CH₂CH₂CH₃.

In some embodiments, the alkyl is a branched alkyl. In some embodiments, R³ and R⁴ are each independently —CH(CH₃) 2 or —CH(CH₃) CH₂CH₃. In some embodiments, R³ and R⁴ are each independently —CH(CH₃)₂.

In some embodiments of the block copolymer of Formula (I), R³ and R⁴ are each independently an optionally substituted C₃-C₁₀ cycloalkyl or aryl. In some embodiments, R³ and R⁴ are each independently an optionally substituted cyclopropyl, cyclobutyl, cyclopentyl, cyclohexyl, or cycloheptyl. In some embodiments, R³ and R⁴ are each independently an optionally substituted phenyl.

In some embodiments of the block copolymer of Formula (I), R³ and R⁴ are taken together with the corresponding nitrogen to which they are attached form an optionally substituted 5 to 7-membered ring. In some embodiments, R³ and R⁴ taken together are —CH₂(CH₂)₂CH₂—, —CH₂(CH₂)₃CH₂—, or —CH₂(CH₂)₄CH₂—. In some embodiments, R³ and R⁴ taken together are —CH₂(CH₂)₂CH₂—. In some embodiments, R³ and R⁴ taken together are —CH₂(CH₂)₃CH₂—. In some embodiments, R³ and R⁴ taken together are —CH₂(CH₂)₄CH₂—.

In some embodiments, the block copolymer of Formula (I) has the structure of Formula (Ia), or a pharmaceutically acceptable salt, solvate, or hydrate thereof:

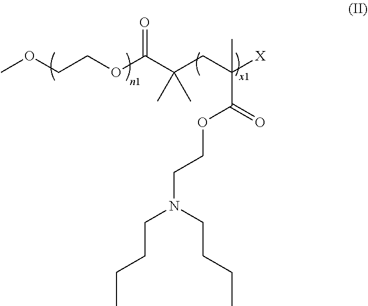

In some embodiments, the block copolymer is a diblock copolymer. In some embodiments, the block copolymer comprises a hydrophilic polymer segment and a hydrophobic segment.

In some embodiments, the hydrophilic polymer segment comprises poly(ethylene oxide) (PEO). In some embodiments, the hydrophilic polymer segment is about 2 kD to about 20 kD in size. In some embodiments, the hydrophilic polymer segment is about 2 kD to about 15 kD in size. In some embodiments, the hydrophilic polymer segment is about 3 kD to about 15 kD in size. In some embodiments, the hydrophilic polymer segment is about 4 kD to about 12 kD in size. In some embodiments, the hydrophilic polymer segment is about 10 kD in size.

In some embodiments, n1 is an integer from 40-500, or any number in between including 40, 80, 115, 230, 340, 460, or 500. In some embodiments, n₁ may range from 80-460, from 115-340, or may be about 230. The value of x1 may range from 4-150, or any number between including 4, 15, 30, 60, 90, 120, or 150. In some embodiments, x1 can range from 15-120, or from 30-90, or may be approximately 60 units. It is understood that any polymer composition will include a distribution of polymers having different number of units for n1 and x1. These compositions will typically have a large number of individual polymers falling within the ranges specified above, although they may include some polymers falling outside of those ranges. In one aspect of the invention, it is contemplated that compositions according to the invention will have a significant fraction of polymers falling with the ranges specified above. Accordingly, it is contemplated that in some cases the average number of units in these polymer compositions will fall within the ranges specified above.

In some embodiments, the block copolymer comprises a hydrophobic polymer segment. In some embodiments, the hydrophobic polymer segment is selected from:

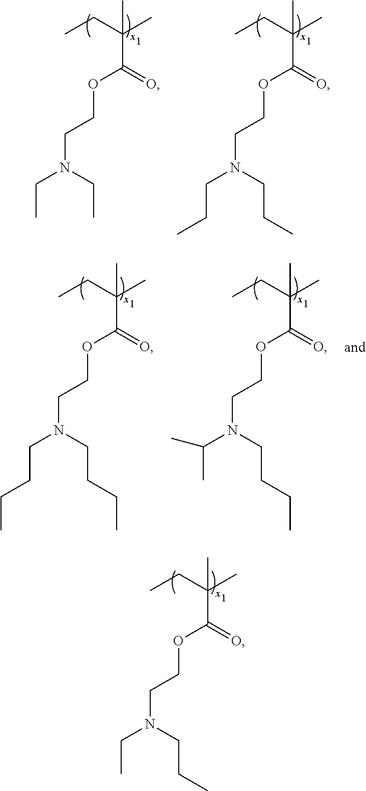

wherein x is about 60-530 or 60-400.

In some embodiments of the block copolymer of Formula (I), R⁵ is hydrogen. In some embodiments, R⁵ is —C(O)CH₃. In some embodiments, R⁵ is acetyl.

In some embodiments of the block copolymer of Formula (I), y₁, is an integer 1-10, 1-9, 1-8, 1-7, 1-6, 1-5, 1-4, 1-3, 1-2, or any range derivable therein. In some embodiments, y₁, is 1, 2, 3, 4, 5, 6, 7, 8, or 9. In some embodiments, y₁, is 1, 2, or 3. In some embodiments, y₁, is 0.

In some embodiments, X is a terminal group. In some embodiments, the terminal capping group is the product of an atom transfer radical polymerization (ATRP) reaction. For example, the terminal capping group may be a halogen, such as —Br, when atom transfer radical polymerization (ATRP) is used. In some embodiments, X is Br. In some embodiments, X is independently —OH. In some embodiments, each X is an acid. In some embodiments, X is —C(O)OH. In some embodiments, X is H. The end group may optionally be further modified following polymerization with an appropriate moiety.

### (ii) Interleukins or Interleukin-Linker-Fc Constructs

Cytokines that are useful in the present invention are capable of being loaded into micelles of the block copolymers described above, and that have an anti-tumor effect. Loading is important because it helps define the dose of cytokines to be transported to the tumor site. The pH difference within the tumor advantageously results in release of the cytokine from the micelle near or within the tumor.

Cytokines that are capable of loading into micelles of the above block copolymer at high levels were interleukins IL-2, IL-15 and IL-21, and to a lesser degree IL-4, IL-7, IL-9, IL-10, IL-12, and IL-28. Conjugation with an antibody fragment is known to improve efficacy of certain cytokines and does not impact the loading efficiency of the cytokine into the micelle. A DNA sequence encoding human IL-2, was expressed, fused with a G4S linker and the Fc region of human IgG1 at the C-terminus.

| Human IL-2 | |
|---|---|
| (SEQ ID: 1) | |
| APTSSSTKKT QLQLEHLLLD LQMILNGINN YKNPKLTRML TFKFYMPKKA TELKHLQCLE | 60 |
| EELKPLEEVL NLAQSKNFHL RPRDLISNIN VIVLELKGSE TTEMCEYADE TATIVEFLNR | 120 |
| WITFCQSIIS TLT | 133 |
| Human IL-4 | |
| (SEQ ID: 2) | |
| HKCDITLQEI IKTLNSLTEQ KTLCTELTVT DIFAASKNTT EKETFCRAAT VLRQFYSHHE | 60 |
| KDTRCLGATA QQFHRHKQLI RFLKRLDRNL WGLAGLNSCP VKEANQSTLE NFLERLKTIM | 120 |
| REKYSKCSS | 129 |
| Human IL-7 | |
| (SEQ ID: 3) | |
| DCDIEGKDGK QYESVLMVSI DQLLDSMKEI GSNCLNNEFN FFKRHICDAN KEGMFLFRAA | 60 |
| RKLRQFLKMN STGDFDLHLL KVSEGTTILL NCTGQVKGRK PAALGEAQPT KSLEENKSLK | 120 |
| EQKKLNDLCF LKRLLQEIKT CWNKILMGTK EHHHHHHH | 158 |
| Human IL-9 | |
| (SEQ ID: 4) | |
| QGCPTLAGIL DINFLINKMQ EDPASKCHCS ANVISCLCLG IPSDNCTRPC FSERLSQMTN | 60 |
| TTMQTRYPLI FSRVKKSVEV LKNNKCPYFS CEQPCNQTTA GNALTELKSL LEIFQKEKMR | 120 |
| GMRGKI | 126 |
| Human IL-10 | |
| (SEQ ID: 5) | |
| SPGQGTQSEN SCTHFPGNLP NMLRDLRDAF SRVKTFFQMK DQLDNLLLKE SLLEDFKGYL | 60 |
| GCQALSEMIQ FYLEEVMPQA ENQDPDIKAH VNSLGENLKT LRLRLRRCHR FLPCENKSKA | 120 |
| VEQVKNAFNK LQEKGIYKAM SEFDIFINYI EAYMTMKIRN | 160 |
| Human IL-12 | |
| (SEQ ID: 6) | |
| RNLPVATPDP GMFPCLHHSQ NLLRAVSNML QKARQTLEFY PCTSEEIDHE DITKDKTSTV | 60 |
| EACLPLELTK NESCLNSRET SFITNGSCLA SRKTSFMMAL CLSSIYEDLK MYQVEFKTMN | 120 |
| AKLLMDPKRQ IFLDQNMLAV IDELMQALNF NSETVPQKSS LEEPDFYKTK IKLCILLHAF | 180 |
| RIRAVTIDRV MSYLNASIWE LKKDVYVVEL DWYPDAPGEM VVLTCDTPEE DGITWTLDQS | 240 |
| SEVLGSGKTL TIQVKEFGDA GQYTCHKGGE VLSHSLLLLH KKEDGIWSTD ILKDQKEPKN | 300 |
| KTFLRCEAKN YSGRFTCWWL TTISTDLTFS VKSSRGSSDP QGVTCGAATL SAERVRGDNK | 360 |
| EYEYSVECQE DSACPAAEES LPIEVMVDAV HKLKYENYTS SFFIRDIIKP DPPKNLQLKP | 420 |
| LKNSRQVEVS WEYPDTWSTP HSYFSLTFCV QVQGKSKREK KDRVFTDKTS ATVICRKNAS | 480 |
| ISVRAQDRYY SSSWSEWASV PCS | 503 |
| Human IL-15 | |
| (SEQ ID: 7) | |
| MHHHHHHNWV NVISDLKKIE DLIQSMHIDA TLYTESDVHP SCKVTAMKCF LLELQVISLE | 60 |
| SGDASIHDTV ENLIILANNS LSSNGNVTES GCKECEELEE KNIKEFLQSF VHIVQMFINT | 120 |
| S | 121 |
| Human IL-21 | |
| (SEQ ID: 8) | |
| QGQDRHMIRM RQLIDIVDQL KNYVNDLVPE FLPAPEDVET NCEWSAFSCF QKAQLKSANT | 60 |
| GNNERIINVS IKKLKRKPPS TNAGRRQKHR LTCPSCDSYE KKPPKEFLER FKSLLQKMIH | 120 |
| QHLSSRTHGS EDS | 133 |
| Fc region of human IgG1 (Glu99-Lys330) | |
| (SEQ ID: 9) | |
| EPKSSDKTHT CPPCPAPELL GGPSVFLFPP KPKDTLMISR TPEVTCVVVD VSHEDPEVKF | 60 |
| NWYVDGVEVH NAKTKPREEQ YNSTYRVVSV LTVLHQDWLN GKEYKCKVSN KALPAPIEKT | 120 |
| ISKAKGQPRE PQVYTLPPSR DELTKNQVSL TCLVKGFYPS DIAVEWESNG QPENNYKTTP | 180 |
| PVLDSDGSFF LYSKLTVDKS RWQQGNVFSC SVMHEALHNH YTQKSLSLSP GK | 232 |
| Linker | |
| (SEQ ID: 10) | |
| GGGGS | 5 |
| Human IL-2 (Ala21-Thr153) - linker-Human IgG1 Fc region | |
| (SEQ ID: 11) | |
| APTSSSTKKT QLQLEHLLLD LQMILNGINN YKNPKLTRML TFKFYMPKKA TELKHLQCLE | 60 |
| EELKPLEEVL NLAQSKNFHL RPRDLISNIN VIVLELKGSE TTFMCEYADE TATIVEFLNR | 120 |
| WITFCQSIIS TLTGGGGSEP KSSDKTHTCP PCPAPELLGG PSVFLFPPKP KDTLMISRTP | 180 |
| EVTCVVVDVS HEDPEVKENW YVDGVEVHNA KTKPREEQYN STYRVVSVLT VLHQDWLNGK | 240 |
| EYKCKVSNKA LPAPIEKTIS KAKGQPREPQ VYTLPPSRDE LTKNQVSLTC LVKGFYPSDI | 300 |
| AVEWESNGQP ENNYKTTPPV LDSDGSFFLY SKLTVDKSRW QQGNVFSCSV MHEALHNHYT | 360 |
| QKSLSLSPGK | 370 |
| Human IL-15 - linker-Human IgG1 Fc region | |
| (SEQ ID: 12) | |
| MHHHHHHNWV NVISDLKKIE DLIQSMHIDA TLYTESDVHP SCKVTAMKCF LLELQVISLE | 60 |
| SGDASIHDTV ENLIILANNS LSSNGNVTES GCKECEELEE KNIKEFLQSF VHIVQMFINT | 120 |
| SGGGGSEPKS SDKTHTCPPC PAPELLGGPS VFLFPPKPKD TLMISRTPEV TCVVVDVSHE | 180 |
| DPEVKFNWYV DGVEVHNAKT KPREEQYNST YRVVSVLTVL HQDWLNGKEY KCKVSNKALP | 240 |
| APIEKTISKA KGQPREPQVY TLPPSRDELT KNQVSLTCLV KGFYPSDIAV EWESNGQPEN | 300 |
| NYKTTPPVLD SDGSFFLYSK LTVDKSRWQQ GNVFSCSVMH EALHNHYTQK SLSLSPGK | 358 |
| Human IL-21 - linker-Human IgG1 Fc region | |
| (SEQ ID: 13) | |
| QKAQLKSANT QGQDRHMIRM RQLIDIVDQL KNYVNDLVPE FLPAPEDVET NCEWSAFSCF | 60 |
| GNNERIINVS IKKLKRKPPS TNAGRRQKHR LTCPSCDSYE KKPPKEFLER FKSLLQKMIH | 120 |
| QHLSSRTHGS EDSGGGGSEP KSSDKTHTCP PCPAPELLGG PSVFLFPPKP KDTLMISRTP | 180 |
| EVTCVVVDVS HEDPEVKENW YVDGVEVHNA KTKPREEQYN STYRVVSVLT VLHQDWLNGK | 240 |
| EYKCKVSNKA LPAPIEKTIS KAKGQPREPQ VYTLPPSRDE LTKNQVSLTC LVKGFYPSDI | 300 |
| AVEWESNGQP ENNYKTTPPV LDSDGSFFLY SKLTVDKSRW QQGNVFSCSV MHEALHNHYT | 360 |
| QKSLSLSPGK | 370 |

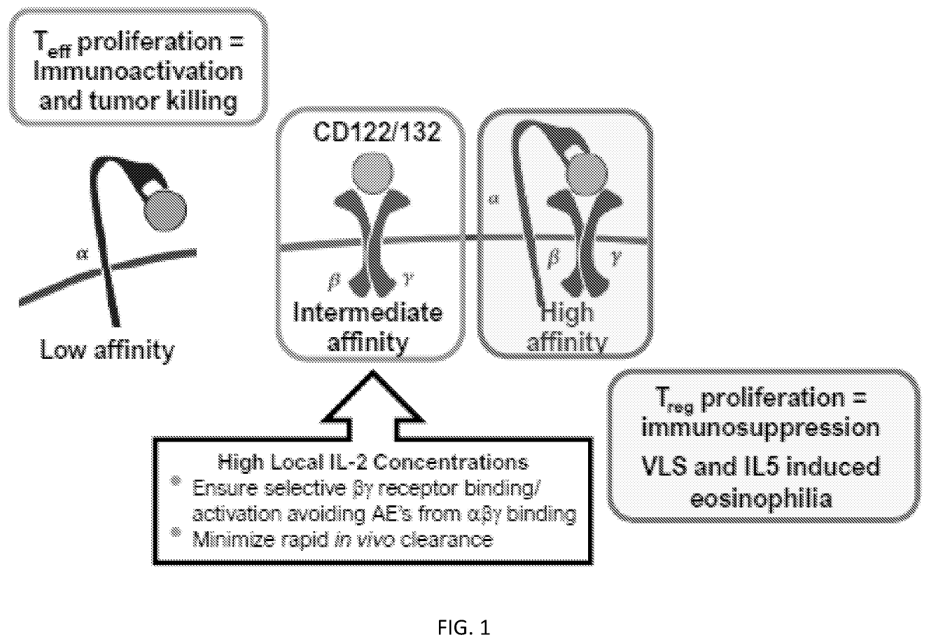

IL-2 receptor biology indicates a requirement for a high local concentration of IL-2. In particular, high local IL-2 concentrations with intermediate affinity binding can ensure selective βγ receptor binding/activation and avoiding of adverse reactions from αβγ binding as well as minimizing rapid in vivo clearance as depicted in FIG. 1. Low affinity binding of IL-2 results in immunoactivation and tumor killing. High affinity can give rise to immunosuppression and VLS and IL5 induced eosinophilia.

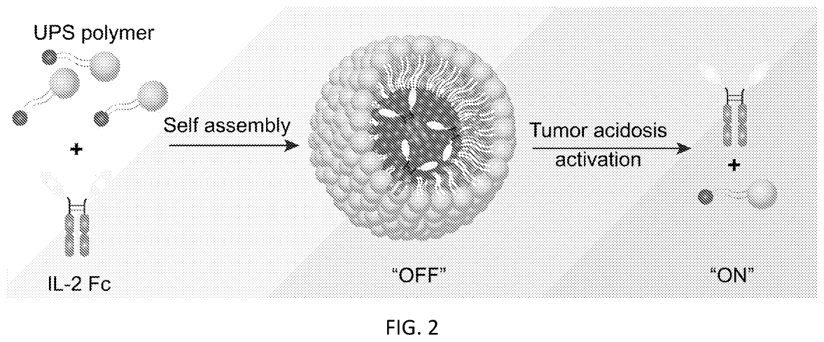

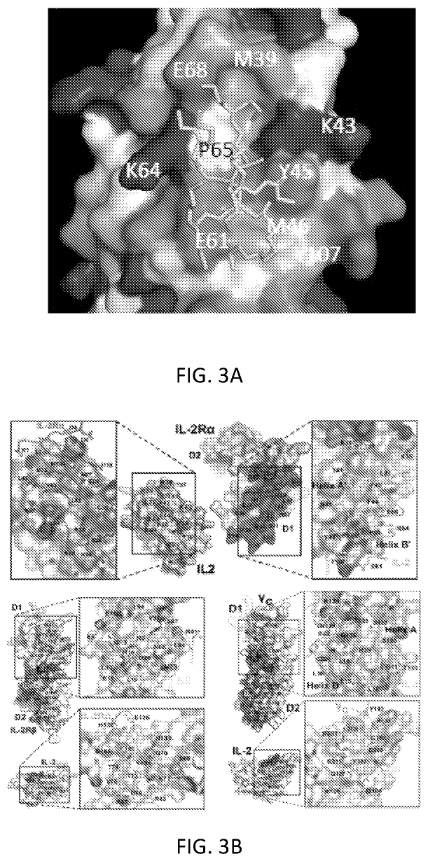

The cytokine encapsulated micelles of the present invention may serve as a non-covalent ON/OFF switch for an Fc-fused cytokine as shown in FIG. 2. The micelles encapsulate IL-2Fc and allow for shaded (“OFF”) activity during circulation. However, in an acidic tumor environment the micelles can release the IL-2-fused antibody fragments allowing for local tumor targeting, or ON activity. The present inventors have discovered that the UPS polymers of the present invention bind to the IL-2Ra domain as shown in FIGS. 3A-B.

The loading of IL-2 in UPS micelles was shown to have a dependency on pH as well as the length of the respective hydrophilic segment and hydrophobic segments for a PDBA polymer. For example, the following PDBA based UPS polymer of formula (II):

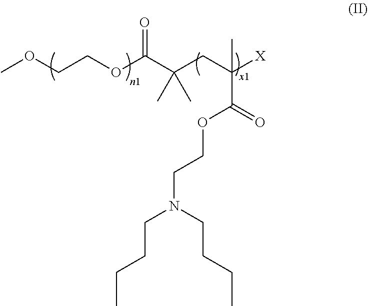

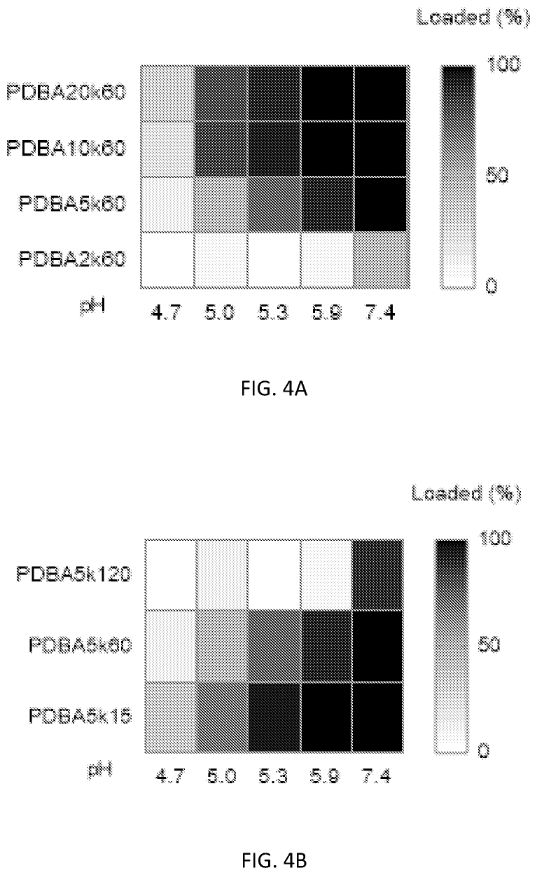

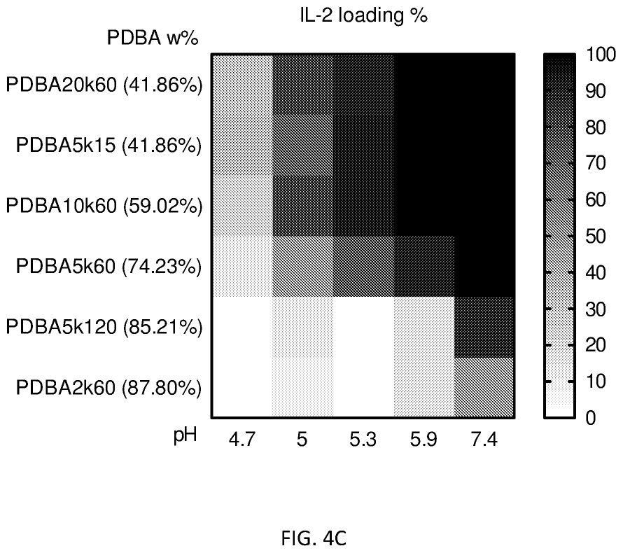

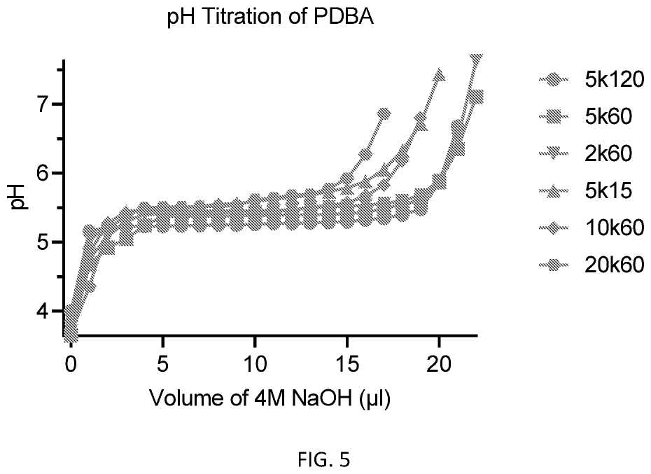

has hydrophilic segments (n₁) and hydrophobic segments (x₁). In the case of PDBA2 k60, the polymer has a number of hydrophilic segments (n₁) corresponding to a hydrophilic segment molecular weight of 2,000 Daltons and 60 (x₁) units. As shown in FIG. 4A, the % loading is highest in the upper right corner of the grid, and lowest in the lower left corner of the grid. Accordingly, the increase in hydrophilic content (higher values of n₁) results in increased loading of IL-2 in the micelle. FIG. 4B shows the effect of different hydrophilic segment length on loading. The lowest percent loading was found in the upper left corner of the grid, while the highest percent loading was found in the lower right portion of the grid. FIG. 4C shows IL-2 loading for PDBA UPS micelles having different hydrophobic and hydrophilic contents over a pH range of 4.7-7.4. The present inventors investigated the effect of hydrophilic and hydrophobic segment molecular weight on pH responsiveness of the for a PDBA based UPS polymer as shown in FIG. 5.

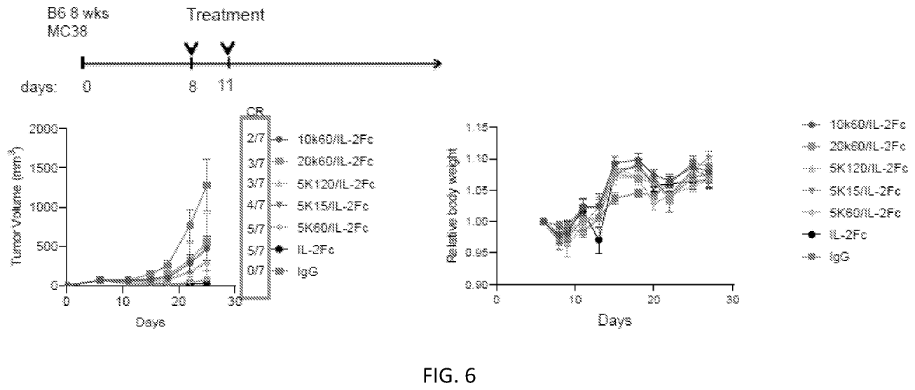

The present inventors found that IL-2Fc with UPS polymer micelles provides potent anti-tumor efficacy that is comparable or even better than free IL-2Fc activity in some groups as shown in FIG. 6. The tumor volume over time is dependent on micelle composition for IL-2Fc. This allows for a tradeoff between avoiding toxicity through higher selectivity and efficacy.

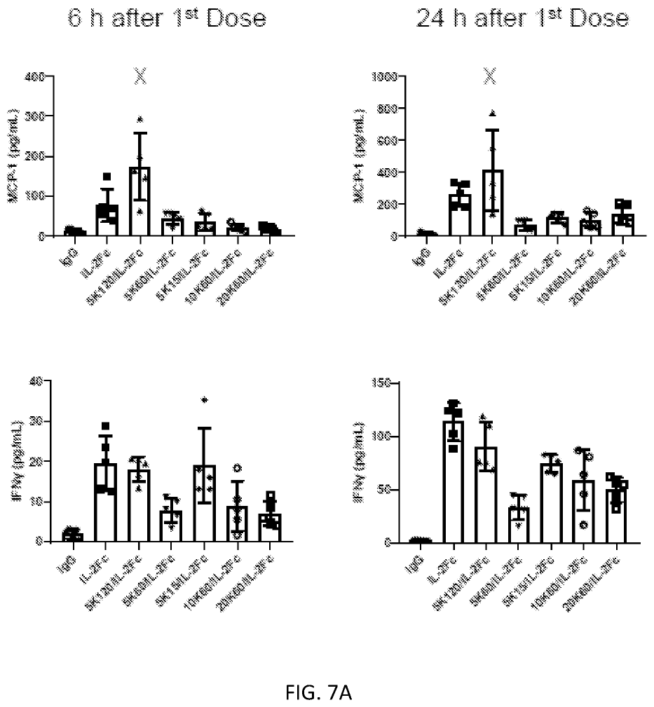

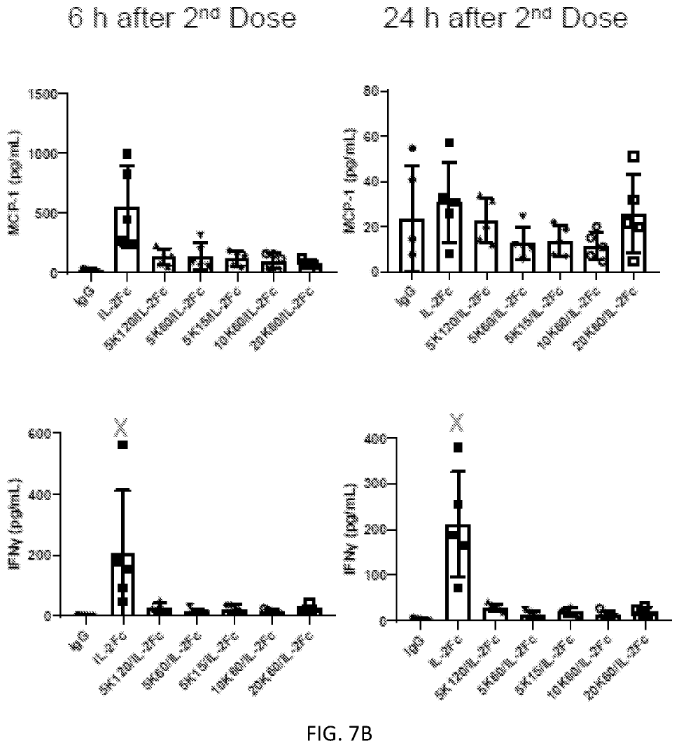

The monocyte hemoattractant protein 1 (MCP-1) expression and IFNγ expression for IL-2Fc-encapsulated UPS polymers were monitored as measurements of toxicity over time as shown in FIGS. 7A-B for MC38 “hot tumor” low dose treatment. The toxicity was studied 6 and 24 hours after a first and second dose. The results show a reduction in toxicity relative to unencapsulated IL-2Fc. The treatment window of efficacy/toxicity is calculated as follows:

$$Window = \frac{Efficacy}{Toxicty}$$

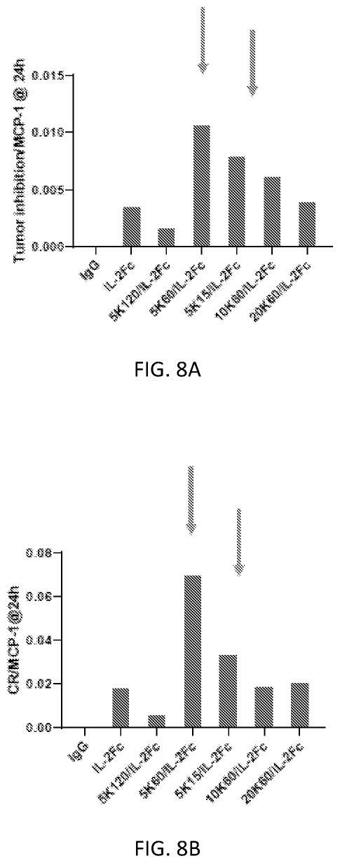

The efficacy is the tumor inhibition, or the number of complete response (CR) as shown in FIG. 8A. The Toxicity is the MCP-1 concentration at 24 hours after the first injection as shown in FIG. 8B. As indicated by the red arrows, the PDBA 5 k60 copolymer encapsulating IL-2Fc showed the greatest level of tumor inhibition compared to other polymers followed by the PDBA 5 k15 IL-2Fc.

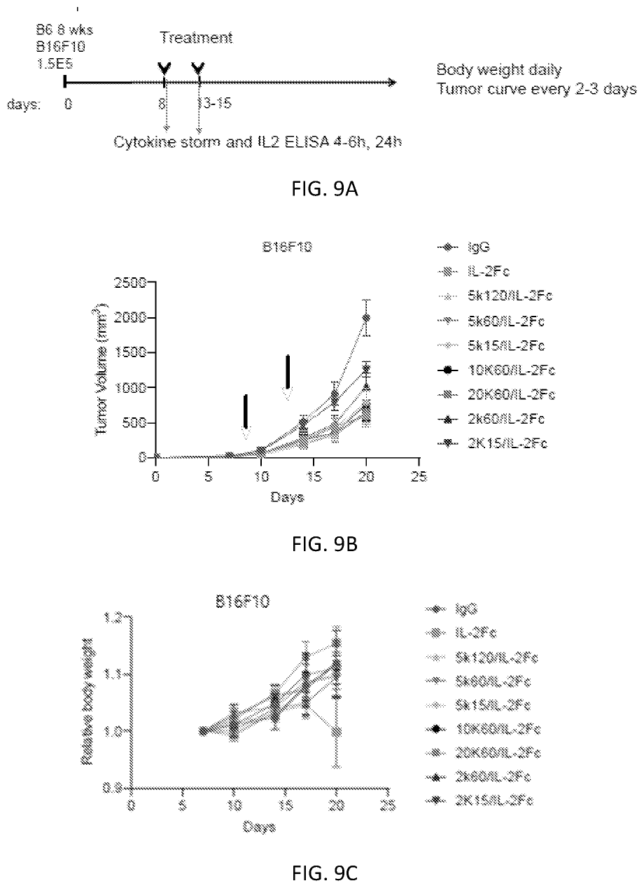

The present inventors found that IL-2Fc encapsulated with UPS polymer micelles provides potent anti-tumor efficacy as indicated by tumor volume that is comparable or even better than free IL-2Fc activity in some groups as shown in FIG. 9A-C. The treatment window for a B16F10 cold tumor, high dose treatment results for cytokine storm and IL-2 ELISA 4-6 h, 24 as shown in FIG. 9A. The tumor volume (antitumor efficacy) for IL-2Fc encapsulated micelles compared to IL-2Fc and IgG is shown in FIG. 9B. The relative body weight as an indicator for toxicity for IL-2Fc encapsulated UPS micelles compared to free IL-2Fc and IgG is shown in FIG. 9C.

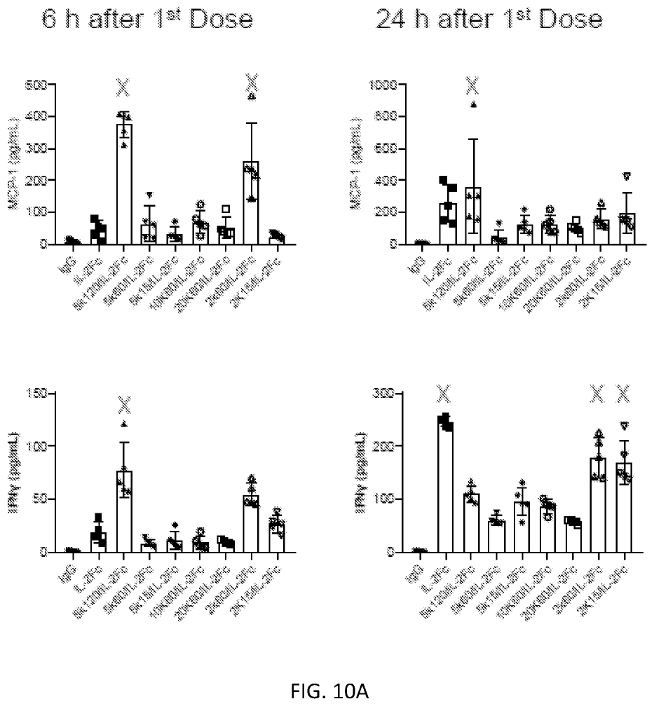

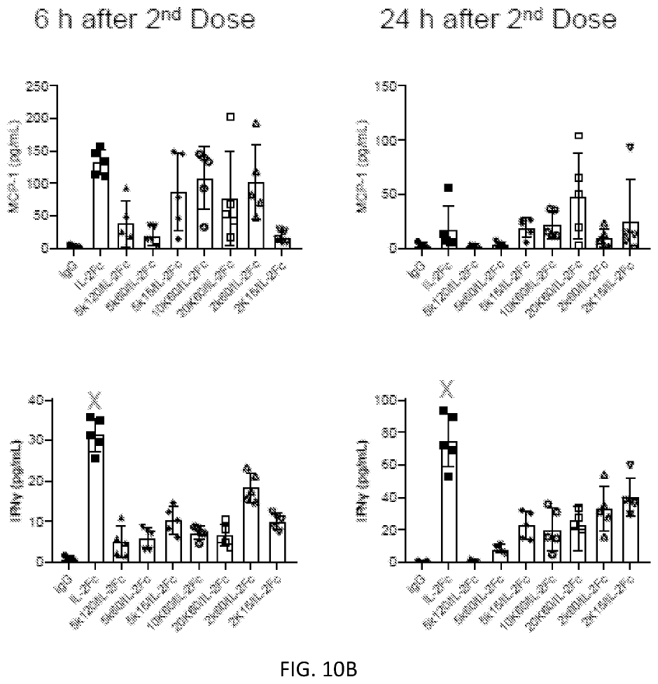

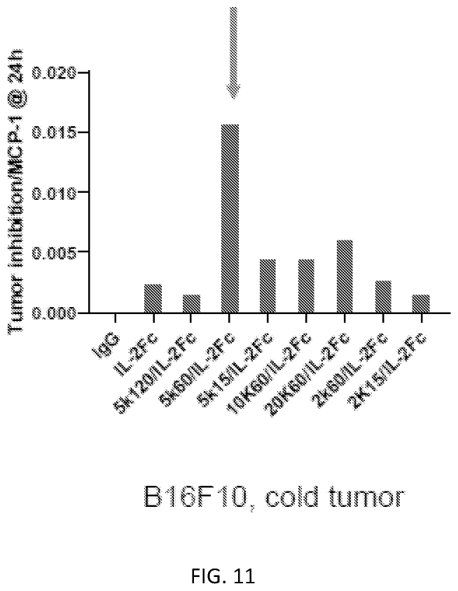

The MCP-1 expression and IFNγ expression for IL-2Fc-encapsulated UPS polymers were monitored as measurements of toxicity over time as shown in FIGS. 10A-B for “cold tumor”, high dose treatment as shown in FIGS. 10A-B. The tumor inhibition for B16F10 k, cold tumor, for various polymer IL-2Fc compositions is shown in FIG. 11. As indicated by the red arrow, the PDBA 5 k60 copolymer encapsulating IL-2Fc showed the greatest level of tumor inhibition compared to other polymers.

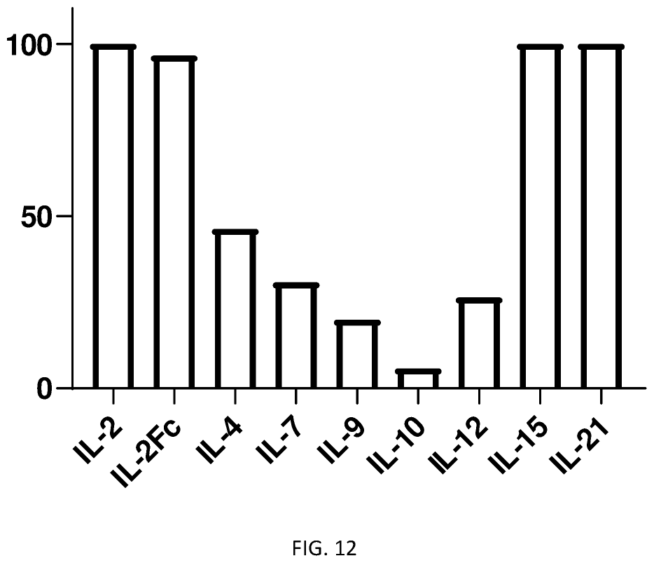

The loading of IL-2, IL-2Fc, IL-4, IL-7, IL-9, IL-10, IL-12, IL-15 and IL-21 into PDBA UPS micelles is shown in FIG. 12. The amount of loading is understood to be driven by the binding the interleukin part. The data show, for example, the both IL-2 and IL-2Fc have excellent binding in the UPS polymer micelles of the present invention. Both IL-15 and IL-21 showed high affinity for loading into the UPS micelles. The present inventors therefore believe that the Fc-bound IL-15 and the Fc-bound IL-21 would also show excellent loading in the polymers of the present invention.

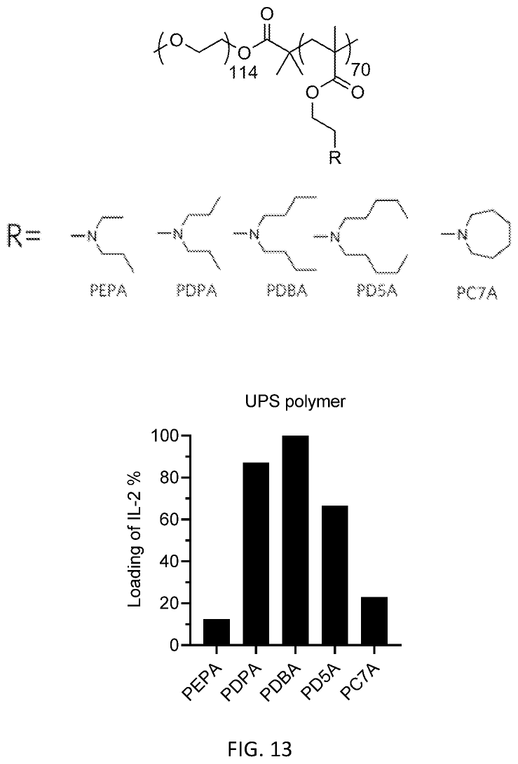

The type of hydrophobic segment used in the UPS micelles was investigated for five types of side chains, PEPA, PDPA, PDBA, PD5A, and PC7A as shown in FIG. 13. The inventors found that PDBA provided the highest relative amount of loading for IL-2, followed by PDPA and then PD5A. From this data it can be seen that selection of PDBA is desirable from a loading perspective. Other factors could influence the decision of which side chain to use, however, and PDPA and PD5A are likely candidates for UPS micelles in addition to PDBA.

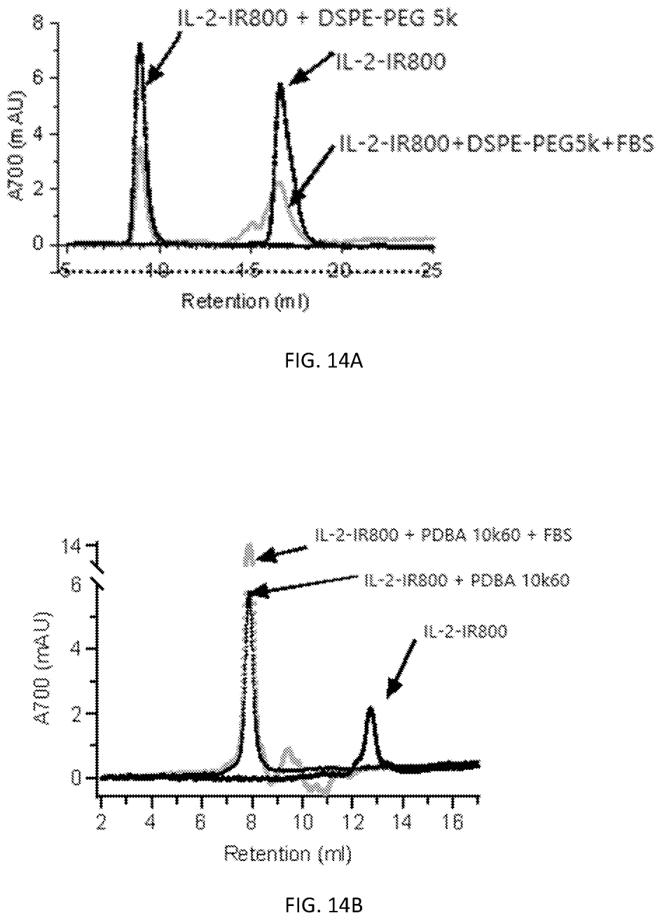

The present inventors investigated the stability of IL-2-R800 encapsulated in various polymers. The FDA-approved pharmaceutical additive 1,2-Distearoyl-sn-glycero-3-phosphorylethanolamine-PEG (DSPE-PEG) was investigated for encapsulation of IL-2 in both water and fetal bovine serum (FBS). As shown in FIG. 14A, DSPE-PEG polymer failed to encapsulate IL-2-IR800 in FBS as it showed separate peaks of approximately similar intensity for both the polymer and IL-2-IR800 even though encapsulation was achieved in water. In contrast, PDBA10 k60 was shown to result in at least nearly complete encapsulation in both FBS and water as shown in FIG. 14B. This demonstrates the present polymers provide encapsulation of interleukins in a physiological environment and provides corroboration that the improvement in toxicity outcomes shown above result from maintenance of encapsulated interleukin post-administration. The distinctive serum stability and pH sensitivity of the IL-2-PEG-PDBA composition makes it a promising formulation for cytokine therapy of cancer.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

The invention claimed is:

1. A micelle composition encapsulating a cytokine active agent comprising:
(i) a block copolymer of Formula (I), or a pharmaceutically acceptable salt, solvate, or hydrate thereof: 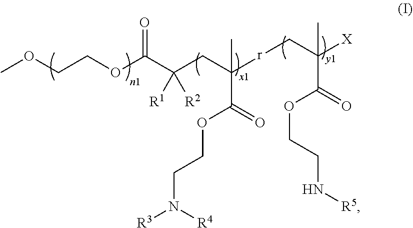
wherein:
n₁ is an integer from 40-500;
x₁ is an integer from 4-150;
y₁ is an integer from 0-10;
X is a halogen, —OH, or —C(O)OH;
R¹ and R² are each independently hydrogen or optionally substituted C₁-C₆ alkyl;
R³ and R⁴ are each independently an optionally substituted C₁-C₆ alkyl, C₃-C₁₀ cycloalkyl or aryl;
or R³ and R⁴ are taken together with the corresponding nitrogen to which they are attached form an optionally substituted 5 to 7-membered ring;
R⁵ is hydrogen or —C(O)CH₃; and
(ii) the cytokine active agent, wherein the cytokine active agent is an interleukin or interleukin-Fc construct.

2. The composition of claim 1, wherein the hydrophobic polymer segment is selected from:

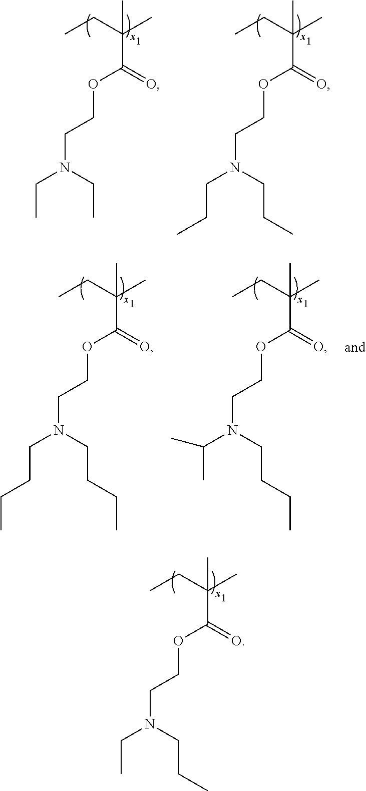

3. The composition of claim 1, wherein the hydrophobic polymer segment is selected from:

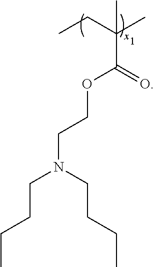

4. The composition of claim 1, wherein n₁ is an integer from 115-340, x₁ is an integer from 30-90, and/or y₁ is 0.

5. The composition of claim 1, wherein the cytokine active agent comprises an amino acid sequence of SEQ ID NOs: 1, 2, 3, 4, 5, 6, 7, 8, or 9 or a homolog that is at least 95% identical to the amino acid sequence of SEQ ID NOs: 1, 2, 3, 4, 5, 6, 7, 8, or 9.

6. The composition of claim 1, wherein the cytokine active agent comprises an Fc region of human IgG.

7. The composition of claim 6, wherein the Fc region comprises an amino acid sequence of SEQ ID NO: 10.

8. The composition of claim 1, wherein the cytokine active agent comprises a linker of SEQ ID: 9.

9. The composition of claim 1, wherein the cytokine active agent comprises an amino acid of SEQ ID NOs: 11, 12, or 13, or a homolog that is at least 95% identical to the amino acid sequence of SEQ ID NOs: 11, 12, or 13.

10. The composition of claim 1, wherein the cytokine active agent comprises an amino acid of SEQ ID NO: 11.

11. A method of treatment of a solid tumor comprising administering a micelle composition encapsulating a cytokine active agent to a patient in need thereof, the composition comprising:
(i) a block copolymer of Formula (I), or a pharmaceutically acceptable salt, solvate, or hydrate thereof: 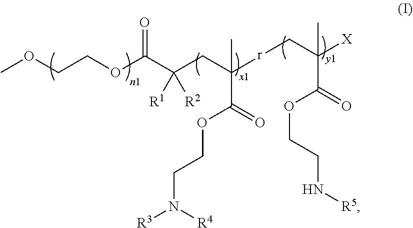
wherein:
n₁ is an integer from 40-500;
x₁ is an integer from 4-150;
y₁ is an integer from 0-10;
X is a halogen, —OH, or —C(O)OH;
R¹ and R² are each independently hydrogen or optionally substituted C₁-C₆ alkyl;
R³ and R⁴ are each independently an optionally substituted C₁-C₆ alkyl, C₃-C₁₀ cycloalkyl or aryl;
or R³ and R⁴ are taken together with the corresponding nitrogen to which they are attached form an optionally substituted 5 to 7-membered ring;
R⁵ is hydrogen or —C(O)CH₃; and
(ii) the cytokine active agent, wherein the cytokine active agent is an interleukin or interleukin-Fc construct.

12. The method of claim 11, wherein the hydrophobic polymer segment is selected from:

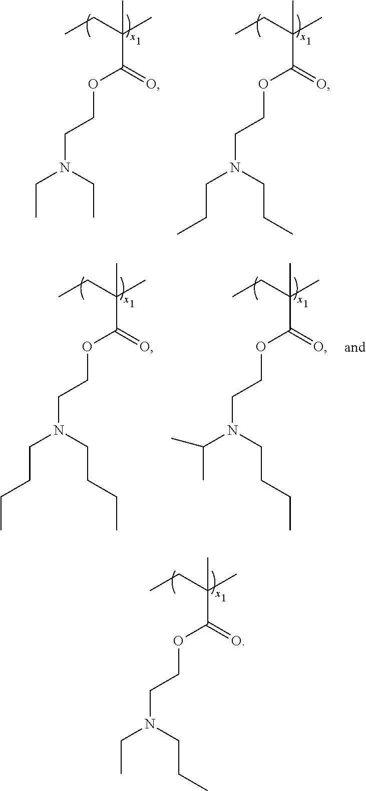

13. The method of claim 11, wherein the hydrophobic polymer segment is selected from:

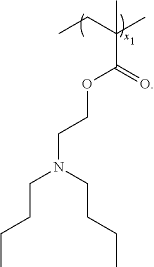

14. The method of claim 11, wherein n₁ is an integer from 115-340, x₁ is an integer from 30-90, and/or y₁ is 0.

15. The method of claim 11, wherein the cytokine active agent comprises an amino acid sequence of SEQ ID NOs: 1, 2, 3, 4, 5, 6, 7, 8, or 9 or a homolog that is at least 95% identical to the amino acid sequence of SEQ ID NOs: 1, 2, 3, 4, 5, 6, 7, 8, or 9.

16. The method of claim 11, wherein the cytokine active agent comprises an Fc region of human IgG.

17. The method of claim 16, wherein the Fc region comprises an amino acid sequence of SEQ ID NO: 10.

18. The method of claim 11, wherein the cytokine active agent comprises a linker of SEQ ID: 9.

19. The method of claim 11, wherein the cytokine active agent comprises an amino acid of SEQ ID NOs: 11, 12, or 13, or a homolog that is at least 95% identical to the amino acid sequence of SEQ ID NOs: 11, 12, or 13.

20. The method of claim 11, wherein the cytokine active agent comprises an amino acid of SEQ ID NO: 11.

21. The method of claim 11, wherein the method comprises administering the composition intratumorally or intravenously.

22. The method of claim 11, wherein the method comprises administering the composition intravenously.

23. The method of claim 11, wherein the composition comprises a pharmaceutically acceptable excipient.
